(19) United States
(12) Patent Application Publication
(10) Pub. No.: US 2017/0181725 A1
(43) Pub. Date: Jun. 29, 2017

(54) JOINT ULTRASOUND IMAGING SYSTEM AND METHOD

(71) Applicant: General Electric Company, Schenectady, NY (US)

(72) Inventors: Xiaodong Han, ShangHai (CN); Menachem Halmann, Wauwatosa, WI (US); Kun Tao, ShangHai (CN); Zhenyu Liu, WuXi (CN); Gang Cheng, ShangHai (CN); Dongqing Chen, Wauwatosa, WI (US); Hong Wang, ShangHai (CN)

(21) Appl. No.: 15/380,493

(22) Filed: Dec. 15, 2016

(30) Foreign Application Priority Data
Dec. 25, 2015 (CN) 20151099970.8

(51) Int. Cl. A61B 8/08; A61B 8/06; A61B 8/00

(52) U.S. Cl. CPC A61B 8/0875; A61B 8/483; A61B 8/54; A61B 8/06; A61B 8/488; A61B 8/4281; A61B 8/4488

(57) ABSTRACT

An ultrasound imaging system includes a scanning assembly, a three-dimensional (3D) image acquisition device, and a controller. The scanning assembly is configured to receive a hand or foot and includes a transducer array and an acoustic coupling fluid. The 3D image acquisition device is configured for obtaining a 3D image of the hand or foot. The controller is configured for automatically adjusting direction or orientation of the transducer array with respect to the hand or foot based on the 3D image of the hand or foot.

### BACKGROUND

The field of the disclosure relates generally to joint ultrasound imaging systems and methods and, more particularly, to an automated joint ultrasound imaging system and method.

In many circumstances, it is beneficial to obtain ultrasound images of joints such as when diagnosing hand or foot injuries or when the joints become inflicted with a disease such as rheumatoid arthritis or gout. With current methods and systems, it is often difficult to capture ultrasound images of such joints in a consistent and efficient manner

Therefore, there is a need for an improved joint ultrasound imaging system and method to address the aforementioned issues.

### BRIEF DESCRIPTION

In accordance with one or more embodiments disclosed herein, an ultrasound imaging system includes a scanning assembly, a three-dimensional (3D) image acquisition device, and a controller. The scanning assembly is configured to receive a hand or foot and includes a transducer array and an acoustic coupling fluid. The 3D image acquisition device is configured for obtaining a 3D image of the hand or foot. The controller is configured for automatically adjusting direction or orientation of the transducer array with respect to the hand or foot based on the 3D image of the hand or foot.

### DRAWINGS

These and other features, aspects, and advantages of the present disclosure will become better understood when the following detailed description is read with reference to the accompanying drawings in which like characters represent like parts throughout the drawings, wherein:

FIG. 1 is a schematic view of a joint ultrasound imaging system in accordance with one exemplary embodiment of the present invention.

FIG. 2 is a schematic view of a joint ultrasound imaging system in accordance with another exemplary embodiment of the present invention.

FIG. 3 is a flow diagram of a method for imaging the joints of the hand or foot in accordance with an exemplary embodiment of the present invention.

FIG. 4 is a schematic view of the actuator in accordance with another exemplary embodiment of the present invention.

FIG. 5 is a schematic view showing the transducer array that is perpendicular to a tangent plane of the hand or foot surface.

### DETAILED DESCRIPTION

In an effort to provide a concise description of these embodiments, not all features of an actual implementation are described in one or more specific embodiments. It should be appreciated that in the development of any such actual implementation, as in any engineering or design project, numerous implementation-specific decisions must be made to achieve the developers' specific goals, such as compliance with system-related and business-related constraints, which may vary from one implementation to another.

Unless defined otherwise, technical and scientific terms used herein have the same meaning as is commonly understood by one of ordinary skill in the art to which this disclosure belongs. The terms “first,” “second,” and the like, as used herein do not denote any order, quantity, or importance, but rather are used to distinguish one element from another. Also, the terms “a” and “an” do not denote a limitation of quantity, but rather denote the presence of at least one of the referenced items. The term “or” is meant to be inclusive and mean either any, several, or all of the listed items. The use of “including,” “comprising,” or “having” and variations thereof herein are meant to encompass the items listed thereafter and equivalents thereof as well as additional items. The terms “connected” and “coupled” are not restricted to physical or mechanical connections or couplings, and can include electrical connections or couplings, whether direct or indirect. The terms “circuit,” “circuitry,” and “controller” may include either a single component or a plurality of components, which are either active and/or passive components and may be optionally connected or otherwise coupled together to provide the described function.

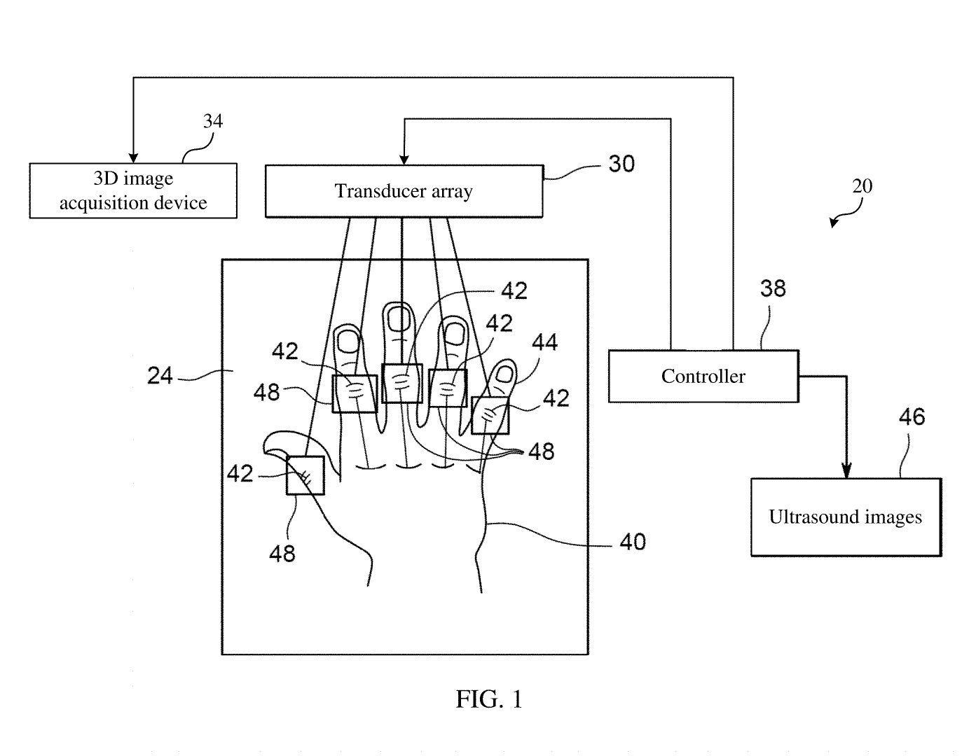

FIG. 1 is a schematic view of a joint ultrasound imaging system 20 in accordance with one exemplary embodiment of the present invention. As described hereafter, the joint ultrasound imaging system 20 consistently captures ultrasound images of joints in an efficient manner. In the example illustrated, the joint ultrasound imaging system 20 includes a scanning assembly comprising a fluid 24, a transducer array 30, a three-dimensional (3D) image acquisition device 34 and a controller 38.

The fluid 24 includes a volume of fluid which serves as an acoustic coupling between a person's hand or foot 40 and the transducer array 30. In one implementation, the fluid 24 includes a bath of water in which the hand or foot 40 is immersed during scanning by the transducer array 30 of the imaging system 20. In other implementations, the fluid 24 may include other forms of a fluid, such as other forms of a liquid, gel or the like, which serve as an acoustic coupling between the hand or foot 40 and the transducer array 30.

The transducer array 30 includes an array of transducers that output signals to facilitate the acquisition of ultrasound images of the joints of the hand or foot 40. In the example illustrated, the transducer array 30 includes quartz crystals, piezoelectric crystals, that change shape in response to the application electrical current so as to produce vibrations or sound waves. Likewise, the impact of sound or pressure waves upon such crystals produce electrical currents. As a result, such crystals are used to send and receive sound waves. Each of the transducers of the transducer array 30 may additionally include a sound absorbing substance to eliminate back reflections and an acoustic lens to focus emitted sound waves. In other embodiments, the acoustic lens may not be included in each of the transducers of the transducer array 30.

The 3D image acquisition device 34 is configured to obtain a 3D image of the hand or foot 40. In the exemplary embodiment, the 3D image acquisition device 34 may include a 3D camera or a 3D coordinate measuring device for example. The controller 38 is configured to identify location of the joint 42 based on the 3D image of the hand or foot 40. As described herein, the joint 42 can be the finger joint of the hand or the toe joint of the foot.

The controller 38 includes a processor or a processing unit that utilizes the identified locations of the finger joints 42 of the hand 40 or the toe joints of the foot to control the operation and/or positioning of the transducer array 30.

The controller 38 controls the operation and/or positioning of the transducer array 30 so as to generate joint ultrasound images 46 of the focus regions 48, which are less than the entire area of the hand or foot 40. Each focus region 48 serves as a window from which the ultrasound images of the joints are taken. In one implementation, the controller 38 controls the operation and/or positioning of the transducer array 30 such that ultrasound pulses are directed at and/or from just those focus regions 48, wherein ultrasound pulses are not directed at regions outside of the focus regions 48 and/or are not received from regions of the hand or foot 40 outside of the focus regions 48. In another implementation, the controller 38 controls the operation and/or positioning of the transducer array 30 such that the transducer array 30 directs ultrasound pulses at and receives ultrasound pulses with respect to portions of the hand or foot 40 that are larger than the focus regions 48, but where the transducer array 30 operates differently with respect to the focus regions 48 as compared to portions of the hand or foot 40 outside of the focus regions 48. For example, in one implementation, the controller 38 controls the transducer array 30 such that a higher density, closer spacing or greater frequency of ultrasound pulses is directed at and received from the focus regions 48 as compared to portions of the hand or foot 40 outside of the focus regions 48. In one implementation, each of the focus regions 48 has an area less than or equal to 9 square centimeters. In one implementation, each of the focus regions 48 has a width of less than or equal to 3 cm.

In one implementation, the controller 38 adjusts the positioning of the transducer array 30 based upon identified locations of the joints 42. For example, in one implementation, the controller 38 outputs control signals which cause an actuator to move the transducer array 30 so as to focus ultrasound imaging on the focus regions 48 extending about the identified locations of the joints 42.

The controller 38 uses the identified locations of the joints 42 to generate or establish a focus region 48 about each of the joints 42, wherein the controller 38 outputs control signal to locate the transducer array 30 in closer proximity to the hand or foot 40 and to move the transducer array 30 between different positions in close proximity to each of the focus regions 48. For example, in one implementation, once the location of the joints 42 have been determined and the focus regions 48 have been generated for each of the determined locations of the joints 42, the controller 38 generates control signals directing an actuator to move the transducer array 30 at a greater first locating speed into close proximity with the hand or foot 40 and adjacent to the focus region 48 over the joint 42 of the person's thumb. In one implementation, the controller 38 generates control signals directing the actuator to move the transducer array 30 at a slower second imaging speed to scan across the focus region 48 over the joint 42 of the person's thumb. Once ultrasound images are acquired for the focus region 48 across the joint 42 of the thumb, the controller 38 generates control signals directing the actuator to move the transducer array 30 at the greater first locating speed to a location adjacent the next focus region to be imaged. This process is repeated until joint ultrasound images 46 have been completed for each of the joints 42 to be imaged. The acquired joint ultrasound images 46 are stored in memory. In one implementation, the acquired joint focus ultrasound images 46 are further displayed.

Because the controller 38 utilizes the determined locations of the joints 42 to form the focus regions 48 and because the controller 38 focuses the transducer array 30 on just those focus regions 48, rather than the entire hand or foot 40, imaging time is reduced and efficiency is increased. As a result, additional time may be spent on such focus regions 48 to increase the amount of imaging data acquired for the joints 42. In implementations where the controller 38 repositions the transducer array 30 at each of the focus regions 48 by moving the transducer array 30 at different speeds, a first greater speed when moving between the focus regions 48 and a second slower speed when scanning across each focus region 48, imaging time is reduced and efficiency is increased.

Although the example illustrated in FIG. 1 illustrates each focus region 48 as being square in shape, in other implementations, each focus region 48 may have other shapes, such as a circular shape, and oval shape or the like. In one implementation, the shape of each focus region 48 corresponds to a general outline of the area of the hand or foot 40 constituting the joint 42. Although the example of FIG. 1 illustrates each focus region 48 as having the same size across each of the joints 42 of the hand or foot 40, in other implementations, the focus regions 48 have different sizes and/or different shapes amongst the different joints 42 of the hand or foot 40.

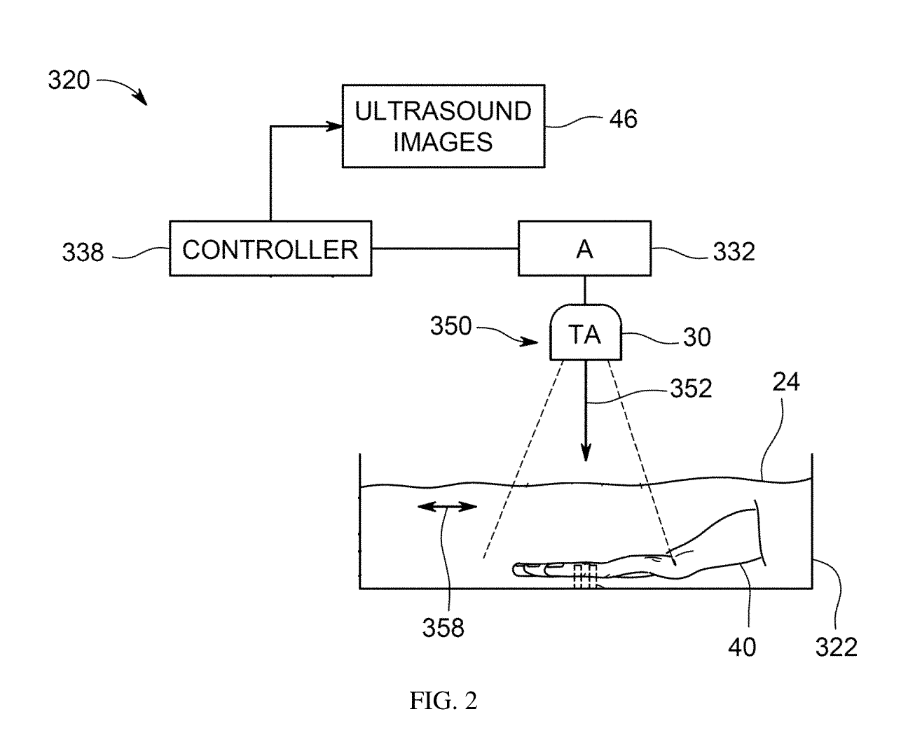

FIG. 2 is a schematic view of a joint ultrasound imaging system 320, another example of the joint ultrasound imaging system 20, in accordance with another exemplary embodiment of the present invention. The joint ultrasound imaging system 320 includes a fluid container 322, fluid 24, a transducer array 30, an actuator 332, and a controller 338. The fluid container 322 includes a receptacle for containing the fluid 24 (described above). The fluid container 322 is sized and configured to receive the hand or foot 40 such that the hand or foot 40 is immersed within the fluid 24.

The actuator 332 includes a mechanism for selectively positioning the transducer array 30 (described above) with respect to the hand or foot 40 submersed in the fluid 24 in response to signals received from the controller 338. In the exemplary embodiment, the actuator 332 includes one or more guide rails slidably or movably supporting the transducer array 30 and one or more linear motors, such as stepper motors, which drive the transducer array 30 to move along one or more guide rails. The actuator 332 further includes one or more rotational motors which drive the transducer array 30 to rotate around one or more guide rails.

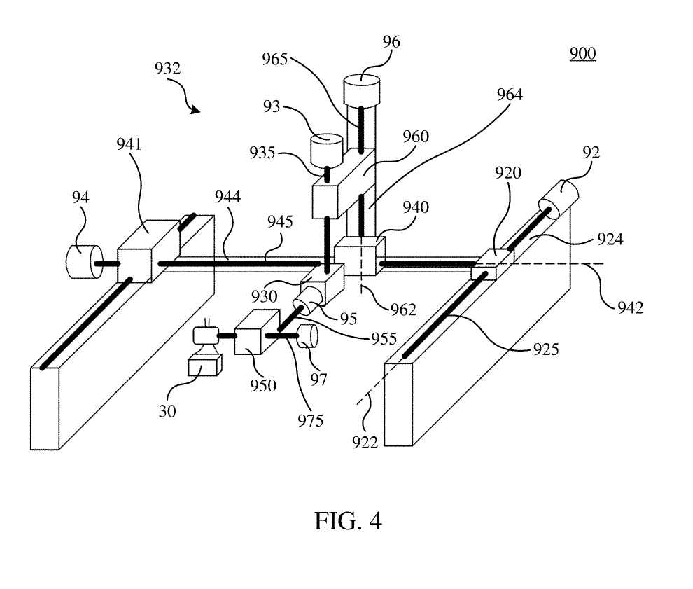

In the exemplary embodiment, the actuator 332 may include one or more guide rails including a horizontal rail, a lateral rail, a vertical rail, one or more linear motors, for example. The lateral rail is perpendicular to the horizontal rail and the vertical rail. One or more linear motors include the motor 92, the motor 94, and the motor 96 (shown in FIG. 4).

The transducer array 30 is moved along the lateral rail extending along a lateral axis by the motor 92 connected to the transducer array 30. The transducer array 30 is also moved along the horizontal rail extending along a horizontal axis, perpendicular to the lateral axis and a vertical axis, by the motor 94 connected to the transducer array 30. The transducer array 30 is moved along the vertical rail extending along the vertical axis by the motor 96 connected to the transducer array 30.

In the exemplary embodiment, the actuator 332 may further include one or more rotational motors, for example. One or more rotational motors include the motor 93, the motor 95, and the motor 97 (shown in FIG. 4).

The transducer array 30 is rotated around the vertical rail by the motor 93 connected to the transducer array 30. The transducer array 30 is also rotated around the lateral rail by the motor 95 connected to the transducer array 30. The transducer array 30 is also rotated around the horizontal rail by the motor 97 connected to the transducer array 30.

The motors 92, 94, 96, 93, 95, and 97 will be described in more detail in FIG. 4.

In yet other embodiments, the actuator 332 may have other configurations.

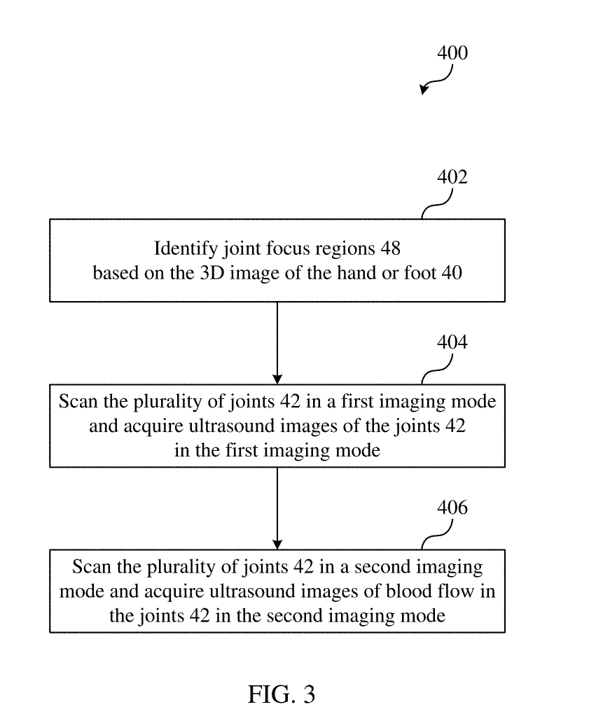

FIG. 3 is a flow diagram of a method 400 for imaging the joints 42 of the hand or foot 40 in accordance with an exemplary embodiment of the present invention. As indicated in block 402 of FIG. 3, the controller 338 is configured to identify focus regions 48 (shown and described above with respect to FIG. 1) based on the 3D image of the hand or foot 40. In detail, a current focus region 48 and a subsequent/next focus region 48 are identified based on the 3D image of the hand or foot 40.

As indicated in block 404 of FIG. 3, the controller 338 is configured to control the actuator 332 to move the transducer array 30, in the direction of arrows 358 to first joint locations at or adjacent to each of the identified focus regions 48. While the transducer array 30 is opposite to each of the identified focus regions 48, the controller 338 directs the transducer array 30 to scan the plurality of joints 42 in a first imaging mode and acquire ultrasound images of the joints 42 in the first imaging mode. In the embodiment, the first imaging mode may be b-mode, for example.

As indicated in block 406 of FIG. 3, the controller 338 is configured to control the actuator 332 to move the transducer array 30, in the direction of arrows 358 to second joint locations at or adjacent to each of the identified focus regions 48. While the transducer array 30 is opposite to each of the identified focus regions 48, the controller 338 directs the transducer array 30 to scan the plurality of joints 42 in a second imaging mode and acquire ultrasound images of blood flow in the joints 42 in the second imaging mode. In the embodiment, the second imaging mode may be a Power Doppler Imaging (PDI) mode or a high resolution PDI mode, for example.

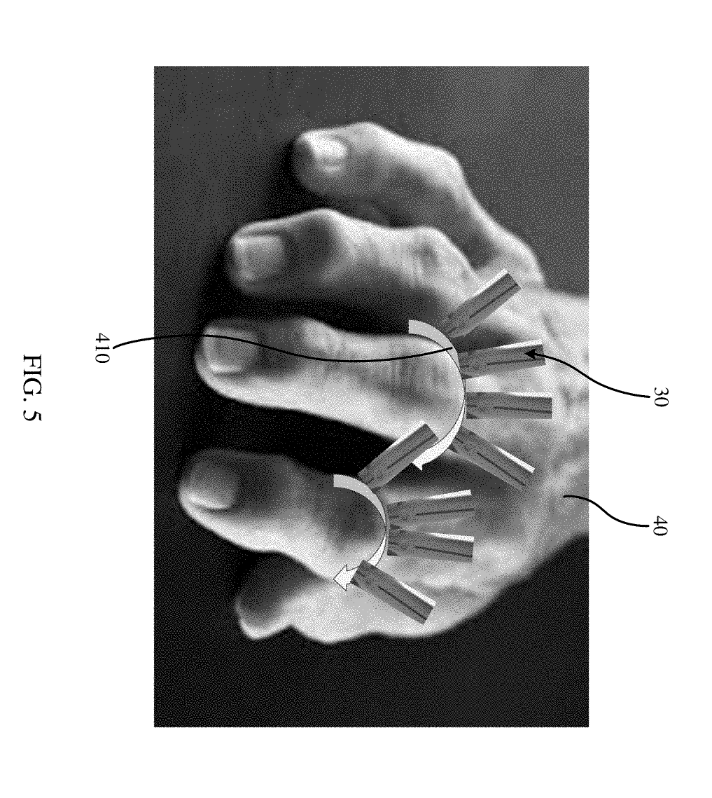

In one embodiment, the controller 338 is further configured to automatically adjust location or position and/or direction or orientation of the transducer array 30 with respect to the hand or foot 40 based on the 3D image of the hand or foot 40, such that the transducer array 30 is perpendicular to a tangent plane 410, as shown in FIG. 5, of the hand or foot surface during a scanning process.

In another embodiment, the controller 338 is further configured to automatically adjust location or position and/or direction or orientation of the transducer array 30 with respect to the hand or foot 40 based on the 3D image of the hand or foot 40, such that ultrasound beam emitted by the transducer array 30 is perpendicular to the hand or foot surface during a scanning process. Therefore, the emitted ultrasound beam will be reflected back to the transducer array 30, but not to other directions.

In one exemplary embodiment, the controller 338 is further configured to automatically adjust location or position of the transducer array 30 with respect to the hand or foot 40 based on the 3D image of the hand or foot 40, such that the transducer array is in close proximity to the hand or foot 40 but without touching the hand or foot 40 during scanning processes. In one embodiment, a distance between the transducer array 30 and the hand or foot 40 may be approximately 2 mm or less, for example.

In another exemplary embodiment, the controller 338 is further configured to automatically adjust location or position of the transducer array 30 with respect to the hand or foot 40 based on the 3D image of the hand or foot 40, such that a distance between the transducer array 30 and the hand or foot 40 is equal to a constant value of approximately 2 mm or less during scanning processes. In one embodiment, the constant value may be 1 mm, for example.

In detail, the ultrasound emitted by the transducer array 30 can real time measure the distance between the transducer array 30 and the hand or foot 40. A planned distance between the transducer array 30 and the hand or foot 40 can be calculated based on the 3D image of the hand or foot 40, because each of the focus regions 48 is identified based on the 3D image of the hand or foot 40. If the measured distance is inconsistent with the planned distance, the controller 338 controls the actuator 332 to adjust the location or position of the transducer array 30, so as to maintain the distance between the transducer array 30 and the hand or foot 40 is equal to the planned distance.

FIG. 4 illustrates a joint ultrasound imaging system 900, an example implementation of the joint ultrasound imaging system 20 of FIG. 1 and the joint ultrasound imaging system 320 of FIG. 2. The ultrasound imaging system 900 is similar to the ultrasound imaging system 320 except that the ultrasound imaging system 900 specifically illustrates the actuator 932, an example of the actuator 832 shown in FIG. 2. Those remaining components or elements of the ultrasound imaging system 900 which correspond to components or elements of the ultrasound imaging system 320 are numbered similarly or are shown in FIG. 2.

As shown in FIG. 4, the actuator 932 includes the motor 92, the motor 94, the motor 96, the motor 93, the motor 95, the motor 97, the horizontal rail 924, the lateral rail 944, the vertical rail 964, threaded shaft 925, and threaded shaft 945.

The horizontal axis 922, the lateral axis 942, and the vertical axis 962 constitute coordinate axes.

The lateral rail 924 extends along the lateral axis 922 and movably supports the first arm 920 for lateral movement along the lateral rail 924. The threaded shaft 925 is connected to the motor 92 and includes external threads that engage internal threads connected to the first arm 920. The motor 92, in response to signals from the controller 338 (shown in FIG. 2), drive the threaded shaft 925 to laterally translate the first arm 920 along the lateral axis 922. Such that the motor 94, the second arm 940; the motor 96, the third arm 960; the motor 93, the fourth arm 930; the motor 95, the fifth arm 950; the motor 97, the sixth arm 970; and the transducer array 30 are also movable along the lateral axis 922.

The horizontal rail 944 extends along the horizontal axis 942 and movably supports the second arm 940 for horizontal movable along the horizontal rail 944. One end of the threaded shaft 945 is connected to the first arm 920 through the sixth arm 941 and the second arm 940, the other end of the threaded shaft 945 is connected to the motor 94. The threaded shaft 945 includes external threads that engage internal threads connected to the second arm 940. The motor 94, in response to signals from the controller 338 (shown in FIG. 2), drive the threaded shaft 945 to horizontally translate the second arm 940 along the horizontal axis 942. Such that the motor 96, the third arm 960; the motor 93, the fourth arm 930; the motor 95, the fifth arm 950; the motor 97, the sixth arm 970; and the transducer array 30 are also movable along the horizontal axis 942.

The vertical rail 964 extends along the vertical axis 962 and movably supports the third arm 960 for vertical movement along the vertical rail 964. The threaded shaft 965 is connected to the motor 96 and includes external threads that engage internal threads connected to the third arm 960. The motor 96, in response to signals from the controller 338 (shown in FIG. 2), drive the threaded shaft 965 to vertically translate the third arm 960 along the vertical axis 962. Such that the motor 93, the fourth arm 930; the motor 95, the fifth arm 950; the motor 97, the sixth arm 970; and the transducer array 30 are also movable along the vertical axis 962.

The actuator 932 further includes a first shaft 935, a second shaft 955, and a third shaft 975.

One end of the first shaft 935 is connected to the motor 93 through the third arm 960. The other end of the first shaft 935 is connected to the fourth arm 930. The motor 93, in response to signals from the controller 338 (shown in FIG. 2), drive the first shaft 935 to cause the fourth arm 930 to rotate around the vertical axis 962. Such that the motor 95, the fifth arm 950; the motor 97, the sixth arm 970 and the transducer array 30 also rotate around the vertical axis 962.

The motor 95 is connected to the fourth arm 930, one end of the second shaft 955 is connected to the motor 95, the other end of the second shaft 955 is connected to the fifth arm 950. The motor 95, in response to signals from the controller 338 (shown in FIG. 2), drive the second shaft 955 to cause the fifth arm 950 to rotate around the lateral axis 922. Such that the motor 97, the sixth arm 970, and the transducer array 30 also rotate around the lateral axis 922.

One end of the third shaft 975 is connected to the motor 97 through the fifth arm 950, the other end of the third shaft 975 is connected to the transducer array 30. The motor 97, in response to signals from the controller 338 (shown in FIG. 2), drive the third shaft 975 to cause the transducer array 30 to rotate around the horizontal axis 942.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure will not be limited to the particular embodiment disclosed as the best mode contemplated for carrying out this disclosure, but that the disclosure will include all embodiments falling within the scope of the appended claims.

1. An ultrasound imaging system, comprising:
a scanning assembly configured to receive a hand or foot and comprising a transducer array and an acoustic coupling fluid;
a three-dimensional (3D) image acquisition device for obtaining a 3D image of the hand or foot; and
a controller for automatically adjusting direction or orientation of the transducer array with respect to the hand or foot based on the 3D image of the hand or foot.

2. The ultrasound imaging system of claim 1, wherein the controller is configured to automatically adjust direction or orientation of the transducer array with respect to the hand or foot based on the 3D image of the hand or foot, such that the transducer array is perpendicular to a tangent plane of the hand or foot surface during a scanning process.

3. The ultrasound imaging system of claim 1, wherein the controller is further configured to automatically adjust direction or orientation of the transducer array with respect to the hand or foot based on the 3D image of the hand or foot, such that ultrasound beam emitted by the transducer array is perpendicular to the hand or foot surface during a scanning process.

4. The ultrasound imaging system of claim 1, wherein the 3D image acquisition device comprises a 3D camera or a 3D coordinate measuring device.

5. The ultrasound imaging system of claim 1, wherein the controller is further configured to automatically adjust the location or position of the transducer array with respect to the hand or foot based on the 3D image of the hand or foot, such that the transducer array is in close proximity to the hand or foot but without touching the hand or foot during scanning processes.

6. The ultrasound imaging system of claim 1, wherein the controller is further configured to automatically adjust location or position of the transducer array with respect to the hand or foot based on the 3D image of the hand or foot, such that a distance between the transducer array and the hand or foot is equal to a constant value during scanning processes.

7. The ultrasound imaging system of claim 1, further comprising:
one or more guide rails; and
one or more rotational motors for controlling the transducer array to rotate around the one or more guide rails;
wherein the controller is further configured to control at least one of the rotational motors to automatically adjust movement of the transducer array with respect to the hand or foot based on the 3D image of the hand or foot.

8. The ultrasound imaging system of claim 7, further comprising:
one or more linear motors for controlling the transducer array to move along the one or more guide rails;
wherein the controller is further configured to control at least one of the rotational motors and the linear motors to automatically adjust movement of the transducer array with respect to the hand or foot based on the 3D image of the hand or foot.

9. The ultrasound imaging system of claim 7, wherein the one or more guide rails comprise a horizontal rail, a lateral rail perpendicular to the horizontal rail, and a vertical rail perpendicular to the horizontal rail and the lateral rail; and
wherein the one or more rotational motors comprise a motor for controlling the transducer array to rotate around the horizontal rail, a motor for controlling the transducer array to rotate around the lateral rail, and a motor for controlling the transducer array to rotate around the vertical rail.

10. The ultrasound imaging system of claim 8, wherein the one or more linear motors comprise a motor for controlling the transducer array to move along the horizontal rail, a motor for controlling the transducer array to move along the lateral rail, and a motor for controlling the transducer array to move along the vertical rail.

11. The ultrasound imaging system of claim 1, wherein the controller is configured to identify location of joints based on the 3D image of the hand or foot, control the transducer array to scan the plurality of joints in a first imaging mode and in a second imaging mode based on the identified location of the joints, and generate ultrasound images of the joints in the first imaging mode and generate ultrasound images of blood flow in the joints in the second imaging mode.

12. The ultrasound imaging system of claim 11, wherein the ultrasound images of the joints comprise b-mode ultrasound images of the joints, the ultrasound images of blood flow in the joints comprise Power Doppler Imaging (PDI) images or high resolution PDI images of the joints.

13. A method of acquiring ultrasound images for a hand or foot examination, the method comprising:
receiving a hand or foot in a scanning assembly including a transducer array and a fluid for providing an acoustic coupling between the transducer array and the hand or foot;
obtaining a 3D image of the hand or foot using a three-dimensional (3D) image acquisition device;
automatically adjusting direction or orientation of the transducer array with respect to the hand or foot based on the 3D image of the hand or foot.

14. The method of claim 13, further comprising:
automatically adjusting direction or orientation of the transducer array with respect to the hand or foot based on the 3D image of the hand or foot, such that the transducer array is perpendicular to a tangent plane of the hand or foot surface during a scanning process.

15. The method of claim 13, further comprising:
automatically adjusting direction or orientation of the transducer array with respect to the hand or foot based on the 3D image of the hand or foot, such that ultrasound beam emitted by the transducer array is perpendicular to the hand or foot surface during a scanning process.

16. The method of claim 13, further comprising:
automatically adjusting the location or position of the transducer array with respect to the hand or foot based on the 3D image of the hand or foot, such that the transducer array is in close proximity to the hand or foot but without touching the hand or foot during scanning processes.

17. The method of claim 13, further comprising:
automatically adjusting the location or position of the transducer array with respect to the hand or foot based on the 3D image of the hand or foot, such that a distance between the transducer array and the hand or foot is equal to a constant value during scanning processes.

18. The method of claim 13, further comprising:
controlling the transducer array to rotate around one or more guide rails using one or more rotational motors; and
controlling at least one of the rotational motors to automatically adjust movement of the transducer array with respect to the hand or foot based on the 3D image of the hand or foot.

19. The method of claim 18, further comprising:
controlling the transducer array to move along the one or more guide rails using one or more linear motors; and
controlling at least one of the rotational motors and the linear motors to automatically adjust movement of the transducer array with respect to the hand or foot based on the 3D image of the hand or foot.

20. The method of claim 18, wherein the one or more guide rails comprise a horizontal rail, a lateral rail perpendicular to the horizontal rail, and a vertical rail perpendicular to the horizontal rail and the lateral rail; and
wherein the one or more rotational motors comprise a motor for controlling the transducer array to rotate around the horizontal rail, a motor for controlling the transducer array to rotate around the lateral rail, and a motor for controlling the transducer array to rotate around the vertical rail.
